SEQUENCE LISTING (1 sequence)

SEQ ID NO: 1 - protein, 406 aa, Homo Sapiens [sequence omitted]

(19) United States
(12) Patent Application Publication
(10) Pub. No.: US 2008/0188400 A1
(43) Pub. Date: Aug. 7, 2008

(54) Methods For Treating Bleeding

(75) Inventors: Mads Ropke, Hellerup (DK); Stephanie J. Lathrop, Mountain View, CA (US)

Correspondence Address: MAXYGEN, INC.;INTELLECTUAL PROPERTY DEPARTMENT, 515 GALVESTON DRIVE, REDWOOD CITY, CA, 94063, US

(73) Assignee: MAXYGEN HOLDINGS LTD.

(21) Appl. No.: 11/912,484

(22) PCT Filed: Apr. 25, 2006
(86) PCT No.: PCT/DK2006/050016

§ 371 Date: Oct. 24, 2007

Related U.S. Application Data
(60) Provisional application No. 60/674,815, filed on Apr. 26, 2005.

(51) Int. Cl. A61K 38/02; A61P 7/04

(52) U.S. Cl. 514/2

(57) ABSTRACT

Methods for the treatment of various bleeding disorders using variants of human Factor VII (hFVII) or activated FVII (FVIIa) having an altered activity compared to 5 recombinant FVIIa with the native human sequence.

### FIELD OF THE INVENTION

The present invention relates to methods of treatment using Factor VII (FVII) or Factor VIIa (FVIIa) polypeptides.

### BACKGROUND OF THE INVENTION

Blood coagulation is a process consisting of a complex interaction of various blood components (or factors) that eventually results in a fibrin clot. Generally, the blood components participating in what has been referred to as the “coagulation cascade” are proenzymes or zymogens, i.e. enzymatically inactive proteins that are converted into an active form by the action of an activator. One of these coagulation factors is FVII.

FVII is a vitamin K-dependent plasma protein synthesized in the liver and secreted into the blood as a single-chain glycoprotein with a molecular weight of 53 kDa (Broze & Majerus, J. Biol. Chem. 1980; 255:1242-1247). The FVII zymogen is converted into an activated form (FVIIa) by proteolytic cleavage at a single site, R152-I153, resulting in two chains linked by a single disulfide bridge. FVIIa in complex with tissue factor (FVIIa complex) is able to convert both factor IX (FIX) and factor X (FX) into their activated forms, followed by reactions leading to rapid thrombin production and fibrin formation (Østerud & Rapaport, Proc Natl Acad Sci USA 1977; 74:5260-5264).

FVII undergoes post-translational modifications, including vitamin K-dependent carboxylation resulting in ten γ-carboxyglutamic acid residues in the N-terminal region of the molecule. Thus, residues number 6, 7, 14, 16, 19, 20, 25, 26, 29 and 35 shown in SEQ ID NO:1 are γ-carboxyglutamic acid residues in the Gla domain important for FVII activity. Other post-translational modifications include sugar moiety attachment at two naturally occurring N-glycosylation sites at position 145 and 322, respectively, and at two naturally occurring O-glycosylation sites at position 52 and 60, respectively.

The gene coding for human FVII (hFVII) has been mapped to chromosome 13 at q34-qter 9 (de Grouchy et al., Hum Genet 1984; 66:230-233). It contains nine exons and spans 12.8 Kb (O'Hara et al., Proc Natl Acad Sci USA 1987; 84:5158-5162). The gene organisation and protein structure of FVII are similar to those of other vitamin K-dependent procoagulant proteins, with exons 1a and 1b encoding for signal sequence; exon 2 the propeptide and Gla domain; exon 3 a short hydrophobic region; exons 4 and 5 the epidermal growth factor-like domains; and exon 6 through 8 the serine protease catalytic domain (Yoshitake et al., Biochemistry 1985; 24:3736-3750).

Reports exist on experimental three-dimensional structures of hFVIIa (Pike et al., Proc Natl Acad Sci USA, 1999; 96:8925-30 and Kemball-Cook et al., J. Struct. Biol., 1999; 127:213-223); of hFVIIa in complex with soluble tissue factor using X-ray crystallographic methods (Banner et al., Nature, 1996; 380:41 and Zhang et al., J. Mol. Biol., 1999; 285: 2089); and of smaller fragments of hFVII (Muranyi et al., Biochemistry, 1998; 37:10605 and Kao et al., Biochemistry, 1999; 38:7097).

Certain protein-engineered variants of FVII have been reported (e.g., Dickinson & Ruf, J Biol Chem, 1997;272:19875-19879; Kemball-Cook et al., J Biol Chem, 1998; 273:8516-8521; Bharadwaj et al., J Biol Chem, 1996; 271:30685-30691; Ruf et al., Biochemistry, 1999; 38:1957-1966).

Reports exist on expression of FVII in BHK or other mammalian cells (WO 92/15686, WO 91/11514 and WO 88/10295) and co-expression of FVII and kex2endoprotease in eukaryotic cells (WO 00/28065).

Commercial preparations of recombinant human FVIIa (rhFVIIa) are sold under the trademark NovoSeven®. NovoSeven® is indicated for the treatment of bleeding episodes in hemophilia A or B patients. NovoSeven® is the only rhFVIIa for effective and reliable treatment of bleeding episodes currently available on the market.

In connection with treatment of uncontrolled bleedings such as trauma it is believed that FVIIa is capable of activating FX to FXa without binding to tissue factor, and this activation reaction is believed to occur primarily on activated blood platelets (Hedner et al. Blood Coagulation & Fibrinolysis, 2000;11;107-111). However, hFVIIa or rhFVIIa has a low activity towards FX in the absence of tissue factor and, consequently, treatment of uncontrolled bleeding, for example in trauma patients, requires relatively high and multiple doses of hFVIIa or rhFVIIa. Therefore, improved FVIIa molecules which possess a high activity toward FX in the absence of tissue factor may be advantageous to treat uncontrolled bleedings more efficiently (to minimize blood loss).

Gla domain variants of FVII/FVIIa have been disclosed in WO 99/20767, U.S. Pat. No. 6,017,882 and WO 00/66753, where some residues located in the Gla domain were identified as being important for phospholipid membrane binding and hence FX activation. In particular, it was found that the residues 10 and 32 were critical and that increased phospholipid membrane binding affinity, and hence increased FX activation, could be achieved by performing the mutations P10Q and K32E. In particular, it was found that FX activation was enhanced as compared to rhFVIIa at marginal coagulation conditions, such as under conditions where a low level of tissue factor is present.

WO 01/58935 discloses a new strategy for developing FVII or FVIIa molecules having inter alia an increased half-life by means of directed glycosylation or PEGylation.

WO 03/093465 discloses FVII or FVIIa variants having certain modifications in the Gla domain and having one or more N-glycosylation sites introduced outside the Gla domain.

WO 2004/029091 discloses FVII or FVIIa variants having certain modifications in the tissue factor binding site.

WO 2004/111242 discloses FVII or FVIIa variants that exhibit an increased phospholipid membrane binding affinity.

The object of the present invention is to provide methods for treating various bleeding conditions using FVII or FVIIa variants having altered activity compared to hFVIIa, for example variants with altered phospholipid membrane binding affinity and/or an altered tissue factor-independent activity.

### BRIEF DISCLOSURE OF THE INVENTION

The present invention relates to methods of treatment using variants of FVII or FVIIa, in particular methods of treating various bleeding disorders using variants of FVIIa having an altered activity compared to FVIIa with the native human sequence.

Additional aspects of the present invention and particular embodiments will be apparent from the description below as well as from the appended claims.

### DETAILED DISCLOSURE OF THE INVENTION

### Definitions

In the context of the present description and claims the following definitions apply:

The term “FVII” or “FVII polypeptide” refers to a FVII molecule provided in single chain form. One example of a FVII polypeptide is the wild-type human FVII (hFVII) having the amino acid sequence shown in SEQ ID NO:1. It should be understood, however, that the term “FVII polypeptide” also covers hFVII-like molecules, such as fragments or variants of SEQ ID NO:1, in particular variants where the sequence comprises at least one, such as up to 15, preferably up to 10, amino acid modifications as compared to SEQ ID NO:1.

The term “FVIIa” or “FVIIa polypeptide” refers to a FVIIa molecule provided in its activated two-chain form. When the amino acid sequence of SEQ ID NO:1 is used to describe the amino acid sequence of FVIla it will be understood that the peptide bond between R152 and I153 of the single-chain form has been cleaved, and that one of the chains comprises amino acid residues 1-152, the other chain comprises amino acid residues 153-406. The polypeptide to be administered in accordance with the methods of the invention will typically be in the activated form, i.e. a variant of human FVIla, although it may in some cases be of interest to administer the polypeptide in the non-activated form, i.e. a variant of human FVII.

The terms “rFVII” and “rFVIla” refer to FVII and FVIla polypeptides produced by recombinant techniques.

The terms “hFVII” and “hFVIla” refer to human wild-type FVII and FVIIa, respectively, having the amino acid sequence shown in SEQ ID NO:1

The terms “rhFVII” and “rhFVIIa” refer to human wild-type FVII and FVIIa, having the amino acid sequence shown in SEQ ID NO:1, produced by recombinant means. An example of rhFVIIa is NovoSeven®.

When used herein, the term “Gla domain” is intended to cover amino acid residues 1 to 45 of SEQ ID NO:1.

Accordingly, the term “position located outside the Gla domain” covers amino acid residues 46-406 of SEQ ID NO:1.

The abbreviations “FX”, “TF” and “TFPI” mean Factor X, Tissue Factor and Tissue Factor Pathway Inhibitor, respectively.

The term “protease domain” is used about residues 153-406 counted from the N-terminus.

The term “catalytic site” is used to mean the catalytic triad consisting of S344, D242 and H193 of the polypeptide variant.

The term “parent” is intended to indicate the FVII or FVIIa molecule from which a FVII or FVIIa variant is derived by way of e.g. substitution, insertion or deletion. Although the parent polypeptide may be any FVII or FVIIa polypeptide, and thus be derived from any origin, e.g. a non-human mammalian origin, it is preferred that the parent polypeptide is hFVII or hFVII.

A “variant” is a polypeptide which differs in one or more amino acid residues from its parent polypeptide, normally in 1-15 amino acid residues (e.g. in 1, 2, 3, 4, 5, 6, 7, 8, 9, 10, 11, 12, 13, 14 or 15 amino acid residues), such as in 1-10 amino acid residues, e.g. in 1-8, 1-6, 1-5 or 1-3 amino acid residues, where the difference between the parent and the variant is e.g. a substitution, insertion and/or deletion. Typically, the variant differs in one or more substitutions from its respective parent. Normally, the parent polypeptide is hFVII or hFVIIa.

The term “conjugate” (or interchangeably “conjugated polypeptide”) is intended to indicate a heterogeneous (in the sense of composite or chimeric) molecule formed by the covalent attachment of one or more polypeptides to one or more non-polypeptide moieties such as polymer molecules, lipophilic compounds, sugar moieties or organic derivatizing agents. Preferably, the conjugate is soluble at relevant concentrations and conditions, i.e. soluble in physiological fluids such as blood. Examples of conjugated polypeptides include glycosylated and/or PEGylated polypeptides.

The term “covalent attachment” or “covalently attached” means that the polypeptide variant and the non-polypeptide moiety are either directly covalently joined to one another, or else are indirectly covalently joined to one another through at least one intervening moiety such as a bridge, spacer, or linkage moiety.

The term “non-polypeptide moiety” is intended to mean a molecule, different from a peptide polymer composed of amino acid monomers and linked together by peptide bonds, which molecule is capable of conjugating to an attachment group of the polypeptide variants described herein. Preferred examples of such molecules include polymer molecules, sugar moieties, lipophilic compounds or organic derivatizing agents. When used in the context of a conjugated variant it will be understood that the non-polypeptide moiety is linked to the polypeptide part of the conjugated variant through an attachment group of the polypeptide. As explained above, the non-polypeptide moiety can be directly or indirectly covalently joined to the attachment group.

A “polymer molecule” is a molecule formed by covalent linkage of two or more monomers, wherein none of the monomers is an amino acid residue, except where the polymer is human albumin or another abundant plasma protein. The term “polymer” may be used interchangeably with the term “polymer molecule”. The term is also intended to cover carbohydrate molecules attached by in vitro glycosylation, i.e. a synthetic glycosylation performed in vitro normally involving covalently linking a carbohydrate molecule to an attachment group of the polypeptide variant, optionally using a cross-linking agent.

The term “sugar moiety” is intended to indicate a carbohydrate-containing molecule comprising one or more monosaccharide residues, capable of being attached to the polypeptide variant (to produce a polypeptide variant conjugate in the form of a glycosylated polypeptide variant) by way of in vivo glycosylation. The term “in vivo glycosylation” is intended to mean any attachment of a sugar moiety occurring in vivo, i.e. during posttranslational processing in a glycosylating cell used for expression of the polypeptide variant, e.g. by way of N-linked and O-linked glycosylation. The exact oligosaccharide structure depends, to a large extent, on the glycosylating organism in question.

An “N-glycosylation site” has the sequence N-X-S/T/C, wherein X is any amino acid residue except proline, N is asparagine and S/T/C is either serine, threonine or cysteine, preferably serine or threonine, and most preferably threonine. Preferably, the amino acid residue in position +3 relative to the asparagine residue is not a proline residue.

An “O-glycosylation site” is the OH-group of a serine or threonine residue.

The term “attachment group” is intended to indicate a functional group of the polypeptide variant, in particular of an amino acid residue thereof or a carbohydrate moiety, capable of attaching a non-polypeptide moiety such as a polymer molecule, a lipophilic molecule, a sugar moiety or an organic derivatizing agent. Useful attachment groups and their matching non-polypeptide moieties are known in the art, e.g. as described in WO 01/58935 and WO 03/093465.

For in vivo N-glycosylation, the term “attachment group” is used to indicate the amino acid residues constituting a N-glycosylation site (with the sequence N-X-S/T/C as mentioned above). Although the asparagine residue of the N-glycosylation site is the one to which the sugar moiety is attached during glycosylation, such attachment cannot be achieved unless the other amino acid residues of the N-glycosylation site are present.

Accordingly, when the non-polypeptide moiety is a sugar moiety and the conjugation is to be achieved by in vivo N-glycosylation, the term “amino acid residue comprising an attachment group for a non-polypeptide moiety” as used in connection with alterations of the amino acid sequence of the polypeptide is to be understood as meaning that one or more amino acid residues constituting an in vivo N-glycosylation site are to be altered in such a manner that a functional in vivo N-glycosylation site is introduced into the amino acid sequence.

The term “amino acid residue” is intended to include any natural or synthetic amino acid residue, and is primarily intended to indicate an amino acid residue contained in the group consisting of the 20 naturally occurring amino acids, i.e. selected from the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues.

The terminology used for identifying amino acid positions is illustrated as follows: G124 indicates that position 124 is occupied by a glycine residue in the amino acid sequence shown in SEQ ID NO:1. G124R indicates that the glycine residue of position 124 has been substituted with an arginine residue. Alternative substitutions are indicated with a “/”, e.g. N145S/T means an amino acid sequence in which asparagine in position 145 is substituted with either serine or threonine. Multiple substitutions are indicated with a “+”, e.g. K143N+N145S/T means an amino acid sequence which comprises a substitution of the lysine residue in position 143 with an asparagine residue and a substitution of the asparagine residue in position 145 with a serine or a threonine residue. Insertion of an additional amino acid residue, e.g. insertion of an alanine residue after G124, is indicated by G124GA. Insertion of two additional alanine residues after G124 is indicated by G124GAA, etc. When used herein, the term “inserted in position X” or “inserted at position X” means that the amino acid residue(s) is (are) inserted between amino acid residue X and X+1. A deletion of an amino acid residue is indicated by an asterix. For example, deletion of the glycine residue in position 124 is indicated by G124*.

Unless otherwise indicated, the numbering of amino acid residues herein is made relative to the amino acid sequence of the hFVII/hFVIIa polypeptide (SEQ ID NO:1).

In addition to the amino acid modifications disclosed herein, it will be understood that the amino acid sequence of the polypeptide variants may, if desired, contain other alterations, i.e. other substitutions, insertions or deletions. These may, for example, include truncation of the N-and/or C-terminus by one or more amino acid residues (e.g. by 1-10 amino acid residues), or addition of one or more extra residues at the N-and/or C-terminus, e.g. addition of a methionine residue at the N-terminus or introduction of a cysteine residue near or at the C-terminus, as well as “conservative amino acid substitutions”, i.e. substitutions performed within groups of amino acids with similar characteristics, e.g. small amino acids, acidic amino acids, polar amino acids, basic amino acids, hydrophobic amino acids and aromatic amino acids.

Examples of such conservative substitutions are shown in the below table.

| | | | | |
|---|---|---|---|---|
| 1 | Alanine (A) | Glycine (G) | Serine (S) | Threonine (T) |
| 2 | Aspartic acid (D) | Glutamic acid (E) | | |
| 3 | Asparagine (N) | Glutamine (Q) | | |
| 4 | Arginine (R) | Histidine (H) | Lysine (K) | |
| 5 | Isoleucine (I) | Leucine (L) | Methionine | Valine (V) |
| | | | (M) | |
| 6 | Phenylalanine (F) | Tyrosine (Y) | Tryptophan | |
| | | | (W) | |

A “polynucleotide” or “nucleotide sequence” as used herein may be of genomic, cDNA, RNA, semisynthetic, synthetic origin, or any combination thereof.

The term “vector” refers to a plasmid or other nucleotide sequences that are capable of replicating within a host cell or being integrated into the host cell genome, and as such, are useful for performing different functions in conjunction with compatible host cells (a vector-host system) to facilitate the cloning of the nucleotide sequence, i.e. to produce useful quantities of the sequence, to direct the expression of the gene product encoded by the sequence and to integrate the nucleotide sequence into the genome of the host cell. The vector will contain different components depending upon the function it is to perform.

“Cell”, “host cell”, “cell line” and “cell culture” are used interchangeably herein and all such terms should be understood to include progeny resulting from growth or culturing of a cell.

“Transformation” and “transfection” are used interchangeably to refer to the process of introducing DNA into a cell.

“Operably linked” refers to the covalent joining of two or more nucleotide sequences, by means of enzymatic ligation or otherwise, in a configuration relative to one another such that the normal function of the sequences can be performed. Generally, “operably linked” means that the nucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

In the context of the present invention the term “modification” or “amino acid modification” is intended to cover replacement of an amino acid side chain, substitution of an amino acid residue, deletion of an amino acid residue or insertion of an amino acid residue.

The term “introduce” refers to introduction of an amino acid residue, in particular by substitution of an existing amino acid residue, or alternatively by insertion of an additional amino acid residue.

The term “remove” refers to removal of an amino acid residue, in particular by substitution of the amino acid residue to be removed by another amino acid residue, or alternatively by deletion (without substitution) of the amino acid residue to be removed.

In the present context, the term “activity” should be understood as the relevant activity associated with the assay in which the activity is actually measured.

Thus, the term “amidolytic activity” is used to mean the activity measured in the “Amidolytic Assay” described herein. In order to exhibit “amidolytic activity” a variant suitable for use in the presently claimed methods, in its activated form, should have at least 10% of the amidolytic activity of rhFVIIa when assayed in the “Amidolytic Assay” described herein. Preferably, the variant, in its activated form, has at least 20% of the amidolytic activity of rhFVIIa, such as at least 30%, e.g. at least 40%, more preferably at least 50%, such as at least 60%, e.g. at least 70%, even more preferably at least 80%, such as at least 90% of the amidolytic activity of rhFVIIa when assayed in the “Amidolytic Assay” described herein. In an interesting embodiment the variant, in its activated form, has substantially the same amidolytic activity as rhFVIIa, such as an amidolytic activity of 75-125% of the amidolytic activity of rhFVIIa.

The term “clotting activity” refers to the activity measured in the “Whole Blood Assay” described herein, i.e. the time needed to obtain clot formation. Thus, a lower clotting time corresponds to a higher clotting activity.

The term “increased clotting activity” is used to indicate that the clotting time of the polypeptide variant is statistically significantly decreased relative to that generated by a reference molecule such as rhFVIIa as determined under comparable conditions and when measured in the “Whole Blood Assay” described herein.

In the present context, the term “activity” is also used in connection with the variants' capability of activating FX to FXa. This activity is also denoted “FX activation activity” or “FXa generation activity” and may be determined in the “TF-independent Factor X Activation Assay” described herein.

The term “increased FX activation activity” or “increased FXa generation activity” is used to indicate that a variant, in its activated form, has a statistically significantly increased capability to activate FX to FXa as compared to a reference molecule such as rhFVIIa. To what extent a variant (in its activated form) has an increased FX activation activity may conveniently be determined in the “TF-independent Factor X Activation Assay” described herein.

The term “immunogenicity” as used in connection with a given substance is intended to indicate the ability of the substance to induce a response from the immune system. The immune response may be a cell or antibody mediated response (see, e.g., Roitt: Essential Immunology (10^{th} Edition, Blackwell) for further definition of immunogenicity). Normally, reduced antibody reactivity will be an indication of reduced immunogenicity. The immunogenicity may be determined by use of any suitable method known in the art, e.g. in vivo or in vitro.

The term “functional in vivo half-life” is used in its normal meaning, i.e. the time at which 50% of the biological activity of the polypeptide is still present in the body/target organ, or the time at which the activity of the polypeptide is 50% of the initial value.

As an alternative to determining functional in vivo half-life, “serum half-life” may be determined, i.e. the time at which 50% of the polypeptide circulates in the plasma or bloodstream prior to being cleared. Determination of serum half-life is often more simple than determining the functional in vivo half-life, and the magnitude of serum half-life is usually a good indication of the magnitude of functional in vivo half-life. Alternative terms to serum half-life include “plasma half-life”, “circulating half-life”, “serum clearance”, “plasma clearance” and “clearance half-life”. The polypeptide is cleared by the action of one or more of the reticuloendothelial systems (RES), kidney, spleen or liver, by tissue factor, SEC receptor or other receptor mediated elimination, or by specific or unspecific proteolysis. Normally, clearance depends on size (relative to the cutoff for glomerular filtration), charge, attached carbohydrate chains, and the presence of cellular receptors for the protein. The functionality to be retained is normally selected from procoagulant, proteolytic or receptor binding activity. The functional in vivo half-life and the serum half-life may be determined by any suitable method known in the art.

The term “increased” as used about the functional in vivo half-life or serum half-life is used to indicate that the relevant half-life of the polypeptide variant is statistically significantly increased relative to that of as reference molecule such as rhFVIIa as determined under comparable conditions (typically determined in an experimental animal, such as rats, rabbits, pigs or monkeys).

The term “AUCᵢᵥ” or “Area Under the Curve when administered intravenously” is used in its normal meaning, i.e. as the area under the activity in serum-time curve, where the polypeptide variant has been administered intravenously, in particular when administered intravenously in rats. Typically, the activity measured is the “clotting activity” as defined above. Once the experimental activity-time points have been determined, the AUCᵢᵥ may conveniently be calculated by a computer program, such as GraphPad Prism 3.01.

It will be understood that in order to make a direct comparison between the AUCᵢᵥ-values of different molecules (e.g. between a variant and a reference molecule such as rhFVIIa) the same amount of activity should be administered. Consequently, the AUCᵢᵥ-values are typically normalized (i.e. corrected for differences in the injected dose) and expressed as AUCᵢᵥ/dose administered.

The term “reduced sensitivity to proteolytic degradation” is primarily intended to mean that the polypeptide variant has reduced sensitivity to proteolytic degradation in comparison to hFVIIa or rhFVIIa as determined under comparable conditions. Preferably, the proteolytic degradation is reduced by at least 10% (e.g. by 10-25% or by 10-50%), such as at least 25% (e.g. by 25-50%, by 25-75% or by 25-100%), more preferably by at least 35%, such as at least 50%, (e.g. by 50-75% or by 50-100%) even more preferably by at least 60%, such as by at least 75% (e.g. by 75-100%) or even at least 90%.

The term “renal clearance” is used in its normal meaning to indicate any clearance taking place by the kidneys, e.g. by glomerular filtration, tubular excretion or degradation in the tubular cells. Renal clearance depends on physical characteristics of the polypeptide, including size (diameter), hydrodynamic volume, symmetry, shape/rigidity, and charge. Renal clearance may be established by any suitable assay, e.g. an established in vivo assay. Typically, renal clearance is determined by administering a labelled (e.g. radiolabelled or fluorescence labelled) polypeptide to a patient and measuring the label activity in urine collected from the patient. Reduced renal clearance is determined relative to a corresponding reference polypeptide, e.g. rhFVIIa, under comparable conditions. Preferably, the renal clearance rate of the polypeptide variant is reduced by at least 50%, preferably by at least 75%, and most preferably by at least 90% compared to rhFVIIa.

The term “hydrophobic amino acid residue” includes the following amino acid residues: Isoleucine (I), leucine (L), methionine (M), valine (V), phenylalanine (F), tyrosine (Y) and tryptophan (W).

The term “negatively charged amino acid residue” includes the following amino acid residues: Aspartic acid (D) and glutamic acid (E).

The term “positively charged amino acid residue” includes the following amino acid residues: Lysine (K), arginine (R) and histidine (H).

The term “treatment” as used herein includes, wherever applicable, not only treatment of an already existing bleeding condition, but also use of the FVII or FVIIa variants for prevention of bleeding episodes. This may for example be the case when the variants are used in circumstances where there may not be an ongoing bleeding, but where there is a risk of problematic bleeding, e.g. in connection with planned surgical procedures, transplantations, or in patients receiving fibrinolytic or anticoagulant drugs.

### Embodiments

### Treatment of Bleeding Caused by Trauma

Trauma may be broadly classified as either blunt or penetrative. Blunt trauma results in internal compression, organ damage and internal hemorrhage, whereas penetrative trauma (as the consequence of an agent penetrating the body and destroying tissue, vessels and organs) results in external hemorrhage.

Hemorrhage as a result of trauma can start a cascade of problems. For example, physiological compensation mechanisms are initiated with initial peripheral and mesenteric vasoconstriction to shunt blood to the central circulation. If circulation is not restored, hypovolemic shock (multiple organ failure due to inadequate perfusion) ensues. Since tissues throughout the body become starved for oxygen, anaerobic metabolism begins. However, the concomitant lactic acid leads to a drop in blood pH and metabolic acidosis develops.

The majority of trauma patients develop hypothermia due to the environmental conditions at the scene, inadequate protection, intravenous fluid administration and ongoing blood loss. Deficiencies in coagulation factors can result from blood loss or transfusions. Meanwhile, acidosis and hypothermia interfere with blood clotting mechanisms. Thus, coagulopathy develops, which in turn may mask surgical bleeding sites and hamper control of mechanical bleeding. Hypothermia, coagulopathy and acidosis are often characterized as the “lethal triad”, as these conditions often lead to multiple organ failure and death.

Trauma may be caused by numerous events. For example, road traffic accidents result in many different types of trauma. While some road traffic accidents are likely to result in penetrative trauma, many road traffic accidents inflict blunt trauma to both head and body. Other major causes of trauma include falls, machinery accidents, gunshot wounds and stab wounds.

One general aspect of the invention relates to methods for treatment of bleeding in patients suffering from various forms of trauma. Particular embodiments of this aspect of the invention are outlined below.

In one embodiment, the invention relates to a method for the treatment of bleeding caused by blunt trauma in a patient, comprising administering to said patient a FVII or FVIIa variant as defined below.

In another embodiment, the invention relates to a method for the treatment of bleeding caused by penetrating trauma in a patient, comprising administering to said patient a FVII or FVIIa variant as defined below.

In another embodiment, the invention relates to a method for the treatment of bleeding caused by crushing trauma in patient, comprising administering to said patient a FVII or FVIIa variant as defined below.

In another embodiment, the invention relates to a method for the treatment of bleeding caused by early stage trauma in a patient, e.g. where the patient has received two bags of blood or less, comprising administering to said patient a FVII or FVIIa variant as defined below.

In another embodiment, the invention relates to a method for the treatment of bleeding caused by late stage trauma in a patient, e.g. where the patient has received at least about 8 bags of blood, such as at least about 10 bags of blood, comprising administering to said patient a FVII or FVIIa variant as defined below.

In another embodiment, the invention relates to a method for the treatment of massive bleeding caused by severe trauma in a patient, comprising administering to said patient a FVII or FVIIa variant as defined below. Massive bleeding can be characterized as a bleeding rate of at least about 150 mL/min or 1.5 mL/min/kg for at least about 20 min. Massive bleeding can also be characterized by the loss of the entire blood volume within 24 hrs (equivalent to about 10 units of packed red blood cells in a 70 kg person) or the loss of 50% of the blood volume within 3 hrs (Martinowitz et al., J Thromb Haemost. (April 2005);3(4):640-8).

In another embodiment, the invention relates to a method for the treatment of bleeding in a coagulopathic patient, comprising administering to said patient a FVII or FVIIa variant as defined below.

In another embodiment, the invention relates to a method for the treatment of bleeding in a patient suffering from pelvic fracture, comprising administering to said patient a FVII or FVIIa variant as defined below.

In another embodiment, the invention relates to a method for the treatment of bleeding in a patient suffering from spleen trauma, comprising administering to said patient a FVII or FVIIa variant as defined below.

In another embodiment, the invention relates to a method for the treatment of bleeding in a patient suffering from a battlefield trauma, comprising administering to said patient a FVII or FVIIa variant as defined below.

In another embodiment, the invention relates to a method for the treatment of patients suffering from hypovolemic shock as a result of blood loss after trauma, comprising administering to said patient a FVII or FVIIa variant as defined below. In addition to hypovolemic shock as a result of decreased circulating blood volume, shock can develop in trauma patients due to activation of inflammatory pathways. Patients with shock are a subset of trauma patients having a particularly high mortality. As mentioned above, shock can be induced by reduced perfusion (hypoperfusion) of various organ systems, resulting in anaerobic metabolism. Indicators of shock include the following:
Tachycardia, defined as a heart rate (HR) above 100 beats per minute (BPM)
Hypotension, defined as a blood pressure (BP) of less than 100 mm Hg
Acidosis, defined as a base deficit (BD) greater than 6 mEq/mL

Shock after trauma and vascular surgery may also activate the thrombomodulin/protein C system by activating the endothelium. Thrombomodulin (TM), a cell surface-expressed glycoprotein that is predominantly synthesized by vascular endothelial cells, is a critical cofactor for thrombin-mediated activation of protein C (PC). Activated PC (APC) inhibits coagulation and promotes fibrinolysis, leading to natural anticoagulant properties. TM, APC, and EPCR have activities that impact coagulation, inflammation, and fibrinolysis. Thus, trauma patients in shock may enter a hypocoagulant state requiring special attention such as higher doses of FVIIa or other pharmacological interventions to reverse this state. Protein C levels decrease after vascular trauma, as PC is activated/cleaved to form APC. Low PC levels (and high TM levels) indicate the presence of shock and can be used as markers for shock and hypoperfusion. Also, plasma concentrations of prothrombin fragment F1+2 or thrombin-antithrombin III-complex (TAT) can be used as markers of thrombin generation.

In an additional embodiment, the invention relates to use of a FVII or FVIIa variant as defined below for the preparation of a medicament for treatment of bleeding in connection with any of the trauma-related indications discussed above.

### Treatment of Intracerebral Hemorrhage or Traumatic Brain Injury

Intracerebral hemorrhage (ICH), also known as brain hemorrhage, intracranial hemorrhage or hemorrhagic stroke, is the most deadly form of stroke. No proven effective treatment for ICH is currently available. Among US and European populations, an estimated 10-15% of all stroke cases are caused by intracerebral hemorrhage, while the figure for Asian populations is estimated to be 20-30%. In addition to high short-term mortality rates, ICH also results in very high rates of severe mental and physical disability among survivors.

ICH can be distinguished from other types of stroke using a CT scan or MRI, after which treatment may be initiated, although until now the available treatment options have only been symptomatic and largely ineffective. If initiated sufficiently early, however, e.g. within about 3-4 hours of the onset of the hemorrhagic stroke, it is contemplated that treatment with the FVII or FVIIa polypeptide variants defined herein may result in significant improvements in terms of increased survival rates and/or decreased disability rates.

The causes of ICH are numerous and can include head trauma (traumatic brain injury, TBI; see below), hypertensive hemorrhage, transformation of prior ischemic infarction (ischemic stroke), metastatic brain tumor, coagulopathy, drug-induced ICH (e.g. induced by cocaine, amphetamine, phenylpropranololamine), arteriovenous malformation, aneurysm, amyloid angiopathy, cavernous angioma, dural arteriovenous fistula and capillary telaniectasias (Harrison's Principles of Internal Medicine, 16^{th} Ed. 2005, McGraw-Hill).

Mayer (Stroke, 2003, 34:224-229) speculated that ultra-early hemostatic treatment of intracerebral hemorrhage (ICH), given within 3-4 hours of onset, may arrest bleeding and minimize hematoma growth after ICH. Mayer et al. (N. Engl. J. Med. 2005, 352(8):777-785) reported that rFVIIa (NovoSeven®) was found to provide a significantly improved neurological and functional outcome and reduced mortality compared to placebo in a clinical study of treatment of ICH. However, it was also reported that treatment with rFVIIa was associated with a small increase in serious thromboembolic adverse events.

It is contemplated that an increased TF-independent activity, optionally with a reduced TF-dependent activity, obtained by use of the FVII or FVIIa variants described herein, may be advantageous over rhFVIIa (NovoSeven®) by reducing or eliminating the risk of thromboembolic events described by Mayer et al. (2005).

Traumatic brain injury (TBI) is another public health problem with high mortality rates and a high frequency of long-term disability. Subarachnoid bleeding leads to increased intracranial pressure on the brain. This can result in reduced blood flow to parts of the brain, leading to ischemia and ultimately infarction. The prevention and treatment of cerebral ischemia are major clinical targets in TBI. As is the case for ICH, the opportunity for treatment (treatment window) is quite short (up to about 3-8 hrs). In the United States alone, there are an estimated 500,000 cases of TBI each year, and also here there is a lack of proven, effective treatments. The available data on clinical trials in head injury is reviewed by Narayan et al., J. Neurotrauma (2002) 19(5):503-557.

Another general aspect of the invention thus relates to methods for the treatment of intracerebral hemorrhage (ICH) or traumatic brain injury (TBI).

One embodiment of this aspect of the invention relates to a method for treatment of primary ICH in a patient, comprising administering to said patient a FVII or FVIIa variant as defined below.

In another embodiment, the invention relates to a method for treatment of ICH secondary to treatment of a patient with tPA (tissue plasminogen activator), comprising administering to said patient a FVII or FVIIa variant as defined below.

In another embodiment, the invention relates to a method for treatment of ICH in an anticoagulated patient, comprising administering to said patient a FVII or FVIIa variant as defined below.

In another embodiment, the invention relates to a method for treatment of ICH caused by subarachnoid hemorrhage in a patient, comprising administering to said patient a FVII or FVIIa variant as defined below.

In another embodiment, the invention relates to a method for treatment of ICH caused by amyloidosis in a patient, comprising administering to said patient a FVII or FVIIa variant as defined below.

In another embodiment, the invention relates to a method for treatment of ICH in a hypertensive patient, comprising administering to said patient a FVII or FVIIa variant as defined below.

In another embodiment, the invention relates to a method for treatment of ICH in a patient suffering from acute cerebellar hemorrhage, comprising administering to said patient a FVII or FVIIa variant as defined below.

In another embodiment, the invention relates to a method for treatment of ICH in a patient suffering from intracranial hemorrhage, comprising administering to said patient a FVII or FVIIa variant as defined below.

In another embodiment, the invention relates to a method for treatment of ICH in a patient suffering from a low level of von Willebrand factor (vWF), comprising administering to said patient a FVII or FVIIa variant as defined below.

In another embodiment, the invention relates to a method for treatment of traumatic brain injury in a patient, comprising administering to said patient a FVII or FVIIa variant as defined below.

Additionally, the invention relates to use of a FVII or FVIIa variant as defined below for the preparation of a medicament for treatment of bleeding in connection with any of the ICH-related indications discussed above or for the treatment of TBI.

### Treatment of Burns

Treatment of severe burns includes excision of the burned area. This procedure often is accompanied by a severe loss of blood. Another general aspect of the invention therefore relates to methods for the treatment of bleeding in connection with burns.

In one embodiment, the invention thus relates to a method for treatment of bleeding in a patient suffering from a burn, in particular a second or third degree burn, comprising administering to said patient a FVII or FVIIa variant as defined below.

Additionally, the invention relates to use of a FVII or FVIIa variant as defined below for the preparation of a medicament for treatment of bleeding in connection with a burn-related indication as discussed above.

### Treatment of Variceal Bleeds

Variceal bleeding is caused by portal hypertension, which is an increase in the pressure within the portal vein that carries blood from the digestive organs to the liver. The increased pressure, which is caused by blockage of blood flow through the liver, causes large veins (varices) to develop across the esophagus and stomach to bypass the blockage. These varices are fragile and can bleed easily. Esophageal varices and resulting variceal bleeding are generally caused by cirrhosis of the liver. Medications may be given to try to shrink the varices, although liver transplantation is generally the only way to cure esophageal varices.

Another aspect of the invention relates to a method for treatment of variceal bleeds in a patient, comprising administering to said patient a FVII or FVIIa variant as defined below, as well as use of the FVII or FVIIa variant for the preparation of a medicament for the treatment of variceal bleeds. For the treatment of variceal bleeds, it may be advantageous to use a combination of administration of the FVII or FVIIa variant together with administration of a beta blocker. In this case, the beta blocker may if desired be administered simultaneously with the FVII or FVIIa variant, or it may be administered separately.

### Treatment of Gastrointestinal Bleeding

Gastrointestinal (GI) bleeding refers to any bleeding in the gastrointestinal tract, i.e. from the mouth to the large bowel, including the esophagus, stomach, and upper and lower gastrointestinal tract. GI bleedings can have a number of different causes, e.g. infections, ulcers, cancer, poisons, medications and alcohol, and can range from minor to massive. Treatment of GI bleeding depends on the location of the bleeding as well as the underlying cause and its severity. For example, in the case of infections, antibiotic treatment will typically be appropriate. In severe cases, it may be necessary to administer fluids intravenously and possibly give blood transfusions.

A further aspect of the invention relates to a method for treatment of gastrointestinal bleeding in a patient, comprising administering to said patient a FVII or FVIIa variant as defined below, as well as use of the FVII or FVIIa variant for the preparation of a medicament for the treatment of gastrointestinal bleeding. For the treatment of gastrointestinal bleeding, it may be advantageous to use a combination of administration of the FVII or FVIIa variant together with administration of an antibiotic. In this case, the antibiotic may if desired be administered simultaneously with the FVII or FVIIa variant, or it may be administered separately.

### Treatment of Surgical Bleeds

Another general aspect of the invention relates to treatment of bleeding in connection with surgery. The surgery in this case may be either a scheduled or acute surgical procedure, and may be any type of surgery on any part of the body. Examples of procedures in which treatment with a FVII or FVIIa variant according to the invention are contemplated to be beneficial are given in the following.

In one embodiment, the invention relates to a method for treatment of bleeding in a patient in connection with spleen tumor removal, splenectomy or spleen biopsy, comprising administering to said patient a FVII or FVIIa variant as defined below.

In another embodiment, the invention relates to a method for treatment of bleeding in a patient in connection with renal tumor removal, complete or partial nephrectomy, or kidney biopsy, comprising administering to said patient a FVII or FVIIa variant as defined below.

In another embodiment, the invention relates to a method for treatment of bleeding in a patient in connection with gastrointestinal surgery (including tumor removal), e.g. surgery on the bowel, duodenum, small intestine, appendix, caecum, colon or rectum, comprising administering to said patient a FVII or FVIla variant as defined below.

In another embodiment, the invention relates to a method for treatment of bleeding in a patient in connection with surgery (including tumor removal) on the upper or lower airways, e.g. the trachea, main bronchus, bronchioles or lung, comprising administering to said patient a FVII or FVIla variant as defined below.

In another embodiment, the invention relates to a method for treatment of bleeding in a patient in connection with cardiac surgery or catheterization, comprising administering to said patient a FVII or FVIIa variant as defined below.

In another embodiment, the invention relates to a method for treatment of bleeding in a patient in connection with surgery (including tumor removal) on the upper or lower limbs, e.g. a hand, arm, shoulder, foot or leg, comprising administering to said patient a FVII or FVIIa variant as defined below.

In another embodiment, the invention relates to a method for treatment of bleeding in a patient in connection with surgery (including tumor removal) on the pelvis, comprising administering to said patient a FVII or FVIIa variant as defined below.

In another embodiment, the invention relates to a method for treatment of bleeding in a patient in connection with tumor surgery, e.g. removal of tumors from the bladder, brain, breast, bowel, cervix, kidney, larynx, liver, lung, esophagus, ovary, pancreas, prostate, skin, stomach, testes or uterus, comprising administering to said patient a FVII or FVIIa variant as defined below.

In another embodiment, the invention relates to a method for treatment of bleeding in a patient in connection with liver surgery, such as liver tumor removal, partial or complete hepatectomy, liver biopsy, gall bladder removal or gall stone removal, comprising administering to said patient a FVII or FVIIa variant as defined below.

In another embodiment, the invention relates to a method for treatment of bleeding in a patient in connection with a biopsy on any part of the body, including a brain biopsy, comprising administering to said patient a FVII or FVIIa variant as defined below.

In another embodiment, the invention relates to a method for treatment of bleeding in a patient in connection with D&C (dilation and curettage), comprising administering to said patient a FVII or FVIIa variant as defined below.

In another embodiment, the invention relates to a method for treatment of bleeding in a patient in connection with a hysterectomy, comprising administering to said patient a FVII or FVIIa variant as defined below.

Additionally, the invention relates to use of a FVII or FVIIa variant as defined below for the preparation of a medicament for the treatment of bleeding in connection with surgery as discussed above.

Treatment of Bleeding in Connection with Transplantation

Another general aspect of the invention relates to treatment of bleeding in connection with transplantation.

In one embodiment, the invention thus relates to a method for treatment of bleeding in connection with a lung transplantation in a patient, comprising administering to said patient a FVII or FVIIa variant as defined below.

In another embodiment, the invention relates to a method for treatment of bleeding in connection with a kidney transplantation in a patient, comprising administering to said patient a FVII or FVIIa variant as defined below.

Additionally, the invention relates to use of a FVII or FVIIa variant as defined below for the preparation of a medicament for the treatment of bleeding in connection with transplantation as discussed above.

### Treatment of Hemorrhage as a Complication to Fibrinolytic Treatment

Thrombolysis, as an initial therapy, reduces the risk of subsequent surgery and improves limb salvage and survival for patients with arterial occlusion. Examples of fibrinolytic drugs used for the treatment of peripheral or central arterial occlusion include Streptokinase, Tissue plasminogen activator, Urinary plasminogen activator, Ancrod, Anistreplase, Plasmin and Reteplase. However, severe bleeding is still a complication of intra-arterial thrombolysis, and the risk of intracranial hemorrhage is 1-2% (Giannini D., Curr Drug Targets Cardiovasc Haematol Disord. 2004;4(3):249-58). In another embodiment, the invention thus relates to a method for treatment of bleeding in a patient receiving a fibrinolytic drug, comprising administering to said patient a FVII or FVIIa variant as defined below, as well as use of a FVII or FVIIa variant as defined below for the preparation of a medicament for the treatment of bleeding as a complication to fibrinolytic treatment.

Treatment of Hemorrhage as a Complication to Treatment with Anticoagulants

Patients receiving long-term anticoagulant therapy (e.g. unfractionated heparin, low-molecular weight heparin, vitamin K antagonists, pentasaccharides, Bivalirudin, Argatroban, Hirudin or Ximelagatran) often experience bleeding as a complication to the anticoagulant treatment. FVIIa variants may be administered to these patients in order to prevent or treat such bleeding complications. Another aspect of the invention thus relates to a method for treatment of bleeding in a patient receiving an anticoagulant drug, comprising administering to said patient a FVII or FVIIa variant as defined below, as well as use of a FVII or FVIIa variant as defined below for the preparation of a medicament for the treatment of bleeding as a complication to anticoagulant treatment. Included in this aspect of the invention are surgical patients receiving anticoagulant therapy.

### Treatment of Postpartum Hemorrhage

Another aspect of the invention relates to a method for treatment of bleeding in a patient caused by postpartum hemorrhage (PPH), i.e. severe bleeding after birth, including hemorrhage associated with caesarian section; hemorrhage associated with vaginal birth; hemorrhage secondary to prior complications including prior molar pregnancy or uterine rupture associated with prior caesarian section, comprising administering to said patient a FVII or FVIIa variant as defined below, as well as use of a FVII or FVIIa variant as defined below for the preparation of a medicament for the treatment of bleeding caused by postpartum hemorrhage.

For the treatment of PPH, it may be advantageous to use a combination of administration of the FVII or FVIIa variant together with administration of a uterotonic drug. In this case, the uterotonic drug may if desired be administered simultaneously with the FVII or FVIIa variant, or it may be administered separately. Further, treatment with the FVII or FVIIa variant, whether with or without treatment with a uterotonic drug, may if desired by accompanied by uterine massage or bimanual compression.

### Treatment of Viral-Induced Hemorrhage

Another general aspect of the invention relates to treatment of viral-induced hemorrhage. It is contemplated that treatment with a FVII or FVIIa variant according to the invention may be useful for hemorrhage caused by any type of virus, for example by Ebola, Marburg, Dengue, Lassa or Crimean-Congo virus.

One embodiment of the invention thus relates to a method for treatment of hemorrhage caused by an Ebola virus infection in a patient, comprising administering to said patient a FVII or FVIIa variant as defined below.

Another embodiment of the invention relates to a method for treatment of hemorrhage caused by a Marburg virus infection in a patient, comprising administering to said patient a FVII or FVIIa variant as defined below.

Another embodiment of the invention relates to a method for treatment of hemorrhage caused by a Dengue virus infection in a patient, comprising administering to said patient a FVII or FVIIa variant as defined below.

Another embodiment of the invention relates to a method for treatment of hemorrhage caused by a Lassa virus infection in a patient, comprising administering to said patient a FVII or FVIIa variant as defined below.

Another embodiment of the invention relates to a method for treatment of hemorrhage caused by a Crimean-Congo virus infection in a patient, comprising administering to said patient a FVII or FVIIa variant as defined below.

Additionally, the invention relates to use of a FVII or FVIIa variant as defined below for the preparation of a medicament for the treatment of bleeding in connection with a viral infection as discussed above.

### Treatment of Thrombocytopenic Patients

Thrombocytopenia encompasses any disorder characterized by a reduced blood platelet (thrombocyte) count resulting from a reduced platelet production and/or an excessive loss of platelets. When the blood platelet count is too low, there is an increased risk of bleeding. There are numerous causes of thrombocytopenia, including decreased bone marrow production of magakaryocytes (e.g. due to marrow infiltration with tumor or fibrosis, or marrow failure induced by e.g. aplasia, hypoplastic anemias, or chemotherapy or other drugs), splenic sequestration of circulating platelets (e.g. splenic enlargement due to tumor infiltration or plenic congestion due to portal hypertension), increased destruction of circulating platelets (e.g. due to vascular prostheses, cardiac valves, disseminated intravascular coagulation (DIC), sepsis, vasculitis, autoantibodies to platelets, drug-associated antibodies, or circulating immune complexes induced by systemic lupus erythematosis, viral agents, bacterial sepsis or idiopathic thrombocytopenic purpura (ITP)), platelet disorders, von Willebrands disease, Bernard-Soulier syndrome, Glanzmann's thrombasthenia, decreased cyclooxygenase activity (drug induced or congenital), granule storage pool defects (acquired or congenital), uremia, platelet coating (e.g. due to penicillin or paraproteins), defective platelet coagulant activity (Scott's syndrome), or thrombocytopenia associated with liver disease such as that caused by hepatitis C or hepatitis B, or caused by IFN-alpha treatment of hepatitis C or hepatitis B.

Another general aspect of the invention relates to treatment of bleeding in connection with thrombocytopenia caused e.g. by any of the conditions discussed above.

In one embodiment, the invention thus relates to a method for treatment of bleeding in connection with thrombocytopenia in a patient, comprising administering to said patient a FVII or FVIla variant as defined below.

Additionally, the invention relates to use of a FVII or FVIla variant as defined below for the preparation of a medicament for the treatment of bleeding in connection with thrombocytopenia, e.g. when caused by any of the conditions discussed above.

### Factor Deficiencies

In another embodiment, the invention relates to a method for treatment of bleeding in a patient with a deficiency of blood factor II, V, VII, X, XI, XII or XIII, or a fibrinogen deficiency, or a patient suffering from dysfibrinogenemia, comprising administering to said patient a FVII or FVIIa variant as defined below, as well as use of the FVII or FVIIa variant for the preparation of a medicament for the treatment of bleeding in connection with a factor deficiency. In this embodiment, the FVII or FVIIa variant described herein may advantageously be administered together with the deficient blood factor other than FVII.

Variants for use in Methods of the Invention FVII or FVIIa variants suitable for use in the methods of the invention generally include at least one modification in the Gla domain and/or at least one amino acid modification that introduces an attachment site for a non-polypeptide moiety, as explained in detail below.

### Modification of the Gla Domain

In one preferred embodiment, the FVII or FVIIa variant includes at least one modification in the Gla domain, in particular at least one modification that results in 30 increased phospholipid membrane binding affinity compared to a similar polypeptide without said modification in the Gla domain. An increased phospholipid membrane affinity is believed to result in a higher local concentration of the activated polypeptide variants in close proximity to other coagulation factors, particularly FX, thus leading to an increase in the rate of activation of FX to FXa and in turn an improved clotting activity. Such modifications in the Gla domain are disclosed e.g. in WO 99/20767, WO 00/66753 and WO 03/093465, and include modifications in one or more of positions 10, 11, 28, 32, 33 and 34 relative to SEQ ID NO:1. Preferably, the variant includes modifications in position 10 and/or 32, optionally together with one or more additional modifications in the Gla domain.

Thus, in one embodiment the variant includes a substitution of a glutamine, a glutamic acid, an aspartic acid or an asparagine residue in position 10, preferably a glutamine residue.

In another embodiment the variant includes a substitution of a glutamic acid or an aspartic acid residue in position 32, preferably a glutamic acid. Preferably, the variant includes substitutions at both of positions 10 and 32, more preferably the substitutions P10Q+K32E.

In another embodiment the variant includes a substitution of a glutamic acid or a phenylalanine residue at position 28.

In another embodiment the variant includes a substitution of a hydrophobic amino acid residue in position 33, the substitution being selected from the group consisting of D331, D33L, D33M, D33V, D33F, D33Y and D33W, in particular D33F.

In another embodiment the variant includes a substitution of a negatively charged residue in position 34, i.e. A34E or A34D, preferably A34E. Alternatively, the variant may include a hydrophobic amino acid residue introduced by substitution in position 34. In this case, the hydrophobic amino acid residue to be introduced in position 34 may be selected from the group consisting of I, L, M, V, F, Y and W, preferably I, L and V, in particular L. When position 34 is modified, the substitution A34E will generally be preferred.

In another embodiment the variant includes an amino acid substitution in position 36. Preferably, the amino acid residue to be introduced by substitution in position 36 is a negatively charged amino acid residue, i.e. R36E or R36D, in particular R36E.

In another embodiment the variant includes an amino acid substitution in position 38, in particular a negatively charged amino acid residue introduced by substitution in position 38, i.e. K38E or K38D, in particular K38E.

In another embodiment the variant includes an insertion of at least one (typically one) amino acid residue between position 3 and 4. The inserted amino acid residue is preferably a hydrophobic amino acid residue. Most preferably the insertion is A3AY.

### Introduction of in Vivo Glycosylation Sites

In another preferred embodiment, the variants used in the methods of the invention comprise one or more modifications that introduce an in vivo N-glycosylation site compared to hFVII with the wild-type sequence. As a result of in vivo attachment of one or more additional oligosaccharide moieties, such variants are generally active in vivo for a longer period of time than recombinant hFVIIa and may be expected to exhibit an overall improved effect in terms of efficiency of clot formation in patients suffering from uncontrolled bleeding events.

As mentioned above, and as is generally known in the art, an N-glycosylation site has the sequence N-X-S/T/C, wherein X is any amino acid residue except proline, N is asparagine and S/T/C is either serine, threonine or cysteine, preferably serine or threonine, and most preferably threonine. Attachment sites for in vivo N-glycosylation can therefore be introduced by modification, typically substitution, of one or two amino acid residues in order to obtain the necessary N-X-S/T/C triplet.

Variants having introduced N-glycosylation sites relative to hFVIIa will typically comprise an 1-5 or 2-5 additional in vivo N-glycosylation sites, such as 1-4 or 1-3 additional in vivo N-glycosylation sites, e.g. 1, 2 or 3 additional in vivo N-glycosylation sites relative to the native sequence. Human FVII has four naturally occurring glycosylation sites at positions N145, N322, S52 and S60, where S52 and S60 are O-glycosylation sites and N145 and N322 are N-glycosylation sites.

It will be understood that in order to prepare a polypeptide comprising one or more sugar moieties covalently attached to one or more in vivo N-glycosylation sites, the polypeptide variant must be expressed in a host cell capable of attaching sugar (oligosaccharide) moieties at the glycosylation site(s) or alternatively subjected to in vitro glycosylation. Preferably, however, the glycosylation is performed in vivo. Examples of glycosylating host cells are listed further below.

Preferably, in vivo N-glycosylation sites are introduced in positions that in hFVIIa comprise an amino acid residue having at least 25% of its side chain exposed to the surface, more preferably in positions comprising an amino acid residue having at least 50% of its side chain exposed to the surface, as defined in WO 01/58935. In general, it is preferred that the in vivo N-glycosylation site is introduced by substitution, although insertion is also contem-plated. It should be understood that when the term “at least 25% (or at least 50%) of its side chain exposed to the surface” is used in connection with introduction of an in vivo N-glycosylation site this term refers to the surface accessibility of the amino acid side chain in the position where the sugar moiety is actually attached. In many cases it will be necessary to introduce a serine or a threonine residue in position +2 relative to the asparagine residue to which the sugar moiety is actually attached (unless, of course, this position is already occupied by a serine or a threonine residue) and these positions where the serine or threonine residues are introduced are allowed to be buried, i.e. to have less than 25% of their side chains exposed to the surface.

Specific and preferred examples of such substitutions creating an in vivo N-glycosylation site include a substitution selected from the group consisting of A51N, G58N, T106N, K109N, G124N, K143N+N145T, A175T, I205S, I205T, V253N, T267N, T267N+S269T, S314N+K316S, S314N+K316T, R315N+V317S, R315N+V317T, K316N+G318S, K316N+G318T, G318N and D334N. More preferably, the in vivo N-glycosylation site is introduced by a substitution selected from the group consisting of A51N, G58N, T106N, K109N, G124N, K143N+N145T, A175T, I205T, V253N, T267N+S269T, S314N+K316T, R315N+V317T, K316N+G318T, G318N and D334N. Still more preferably, the in vivo N-glycosylation site is introduced by a substitution selected from the group consisting of T106N, I205T and V253N.

In one embodiment, only one in vivo N-glycosylation site has been introduced by substitution. In another embodiment, two or more in vivo N-glycosylation sites have been introduced by substitution. Examples of preferred substitutions creating two in vivo N-glycosylation sites include substitutions selected from the group consisting of A51N+G58N, A51N+T106N, A51N+K109N, A51N+G124N, A51N+K143N+N145T, A51N+A175T, A51N+I205T, A51N+V253N, A51N+T267N+S269T, A51N+S314N+K316T, A51N+R315N+V317T, A51N+K316N+G318T, A51N+G318N, A51N+D334N, G58N+T106N, G58N+K109N, G58N+G124N, G58N+K143N+N145T, G58N+A175T, G58N+I205T, G58N+V253N, G58N+T267N+S269T, G58N+S314N+K316T, G58N+R315N+V317T, G58N+K316N+G318T, G58N+G318N, G58N+D334N, T106N+K109N, T106N+G124N, T106N+K143N+N145T, T106N+A175T, T106N+I205T, T106N+V253N, T106N+T267N+S269T, T106N+S314N+K316T, T106N+R315N+V317T, T106N+K316N+G318T, T106N+G318N, T106N+D334N, K109N+G124N, K109N+K143N+N145T, K109N+A175T, K109N+I205T, K109N+V253N, K109N+T267N+S269T, K109N+S314N+K316T, K109N+R315N+V317T, K109N+K316N+G318T, K109N+G318N, K109N+D334N, G124N+K143N+N145T, G124N+A175T, G124N+I205T, G124N+V253N, G124N+T267N+S269T, G124N+S314N+K316T, G124N+R315N+V317T, G124N+K316N+G318T, G124N+G318N, G124N+D334N, K143N+N145T+A175T, K143N+N145T+I205T, K143N+N145T+V253N, K143N+N145T+T267N+S269T, K143N+N145T+S314N+K316T, K143N+N145T+R315N+V317T, K143N+N145T+K316N+G318T, K143N+N145T+G318N, K143N+N145T+D334N, A175T+I205T, A175T+V253N, A175T+T267N+S269T, A175T+S314N+K316T, A175T+R315N+V317T, A175T+K316N+G318T, A175T+G318N, A175T+D334N, I205T+V253N, I205T+T267N+S269T, I205T+S314N+K316T, I205T+R315N+V317T, I205T+K316N+G318T, I205T+G318N, I205T+D334N, V253N+T267N+S269T, V253N+S314N+K316T, V253N+R315N+V317T, V253N+K316N+G318T, V253N+G318N, V253N+D334N, T267N+S269T+S314N+K316T, T267N+S269T+R315N+V317T, T267N+S269T+K316N+G318T, T267N+S269T+G318N, T267N+S269T+D334N, S314N+K316T+R315N+V317T, S314N+K316T+G318N, S314N+K316T+D334N, R315N+V317T+K316N+G318T, R315N+V317T+G318N, R315N+V317T+D334N and G318N+D334N. More preferably, the substitutions are selected from the group consisting of T106N+A175T, T106N+I205T, T106N+V253N, T106N+T267N+S269T, A175T+I205T, A175T+V253N, A175T+T267N+S269T, I205T+V253N, I205T+T267N+S269T and V253N+T267N+S269T, even more preferably from the group consisting of T106N+I205T, T106N+V253N and I205T+V253N.

In a further embodiment, three or more in vivo N-glycosylation sites have been introduced by substitution. Examples of preferred substitutions creating three in vivo N-glycosylation sites include substitutions selected from the group consisting of I205T+V253N+T267N+S269T and T106N+I205T+V253N.

It should further be noted that the positions to be modified are preferably selected from parts of the FVII or FVIIa molecule that are located outside the active site region (where the active site region is defined as any residues having at least one atom within 10 Å of any atom in the catalytic triad comprising residues H193, D242, S344) and the ridge of the active site binding cleft, at least when active FVII or FVIIa variants having procoagulant properties are desired, since conjugation in these regions may result in inactivation or reduced activity of the resulting conjugate. In contrast, if inactive FVII or FVIIa variants having properties suitable for use as an anticoagulant are desired, it may be advantageous to introduce one or more sites for N-glycosylation in these regions. These two regions are identified in WO 01/58935 and WO 03/093465 as follows:

Active site region: I153, Q167, V168, L169, L170, L171, Q176, L177, C178, G179, G180, T181, V188, V189, S190, A191, A192, H193, C194, F195, D196, K197, I198, W201, V228, I229, I230, P231, S232, T233, Y234, V235, P236, G237, T238, T239, N240, H241, D242, I243, A244, L245, L246, V281, S282, G283, W284, G285, Q286, T293, T324, E325, Y326, M327, F328, D338, S339, C340, K341, G342, D343, S344, G345, G346, P347, H348, L358, T359, G360, I361, V362, S363, W364, G365, C368, V376, Y377, T378, R379, V380, Q382, Y383, W386, L387, L400 and F405.

Ridge of the active site binding cleft: N173, A175, K199, N200, N203, D289, R290, G291, A292, P321 and T370.

Variants having a Modification in the Gla Domain and an Introduced N-glycosylation Site

In a particularly preferred aspect, the methods of the invention are performed using a FVII or FVIIa variant having at least one modification in the Gla domain and at least one introduced in vivo N-glycosyation site as described in the respective sections above.

As indicated above, the variant preferably includes substitutions at one or both of positions 10 and 32, preferably both of these position, and more preferably the substitutions P10Q+K32E. In one preferred embodiment, substitutions in positions 10 and 32 are combined with one or more additional substitutions in the Gla domain, e.g. in position 34, 36 and/or 38. In another preferred embodiment, substitutions in positions 10 and 32 are combined with one or more introduced in vivo N-glycosylation sites. In a further preferred embodiment, substitutions in positions 10 and 32 are combined with one or more additional substitutions in the Gla domain, e.g. in position 34, 36 and/or 38, and with one or more introduced in vivo N-glycosylation sites.

Specific examples of variants having multiple substitutions in the Gla domain include:

P10Q+K32E;

P10Q+K32E+A34E;

P10Q+K32E+R36E;

P10Q+K32E+K38E;

P10Q+K32E+A34E+R36E;

P10Q+K32E+R36E+K38E;

P10Q+K32E+A34E+K38E;

P10Q+K32E+A34E+R36E+K38E;

P10Q+K32E+A34L;

P10Q+K32E+A34L+R36E;

P10Q+K32E+A34L+K38E; and

P10Q+K32E+A34L+R36E+K38E.

Preferred variants having multiple substitutions in the Gla domain include:

P10Q+K32E;

P10Q+K32E+A34E;

P10Q+K32E+R36E;

P10Q+K32E+A34E+R36E.

Specific examples of “combined” variants having multiple substitutions in the Gla domain and at least one introduced N-glycosylation site include:

P10Q+K32E+T106N;

P10Q+K32E+A34E+T106N;

P10Q+K32E+R36E+T106N;

P10Q+K32E+A34E+R36E+T106N;

P10Q+K32E+I205T;

P10Q+K32E+A34E+I205T;

P10Q+K32E+R36E+I205T;

P10Q+K32E+A34E+R36E+I205T;

P10Q+K32E+V253N;

P10Q+K32E+A34E+V253N;

P10Q+K32E+R36E+V253N;

P10Q+K32E+A34E+R36E+V253N;

P10Q+K32E+T106N+I205T;

P10Q+K32E+A34E+T106N+I205T;

P10Q+K32E+R36E+T106N+I205T;

P10Q+K32E+A34E+R36E+T106N+I205T;

P10Q+K32E+T106N+V253N;

P10Q+K32E+A34E+T106N+V253N;

P10Q+K32E+R36E+T106N+V253N;

P10Q+K32E+A34E+R36E+T106N+V253N;

P10Q+K32E+I205T+V253N;

P10Q+K32E+A34E+I205T+V253N;

P10Q+K32E+R36E+I205T+V253N;

P10Q+K32E+A34E+R36E+I205T+V253N;

P10Q+K32E+T106N+I205T+V253N;

P10Q+K32E+A34E+T106N+I205T+V253N;

P10Q+K32E+R36E+T106N+I205T+V253N;

P10Q+K32E+A34E+R36E+T106N+I205T+V253N;

P10Q+K32E+A34L+T106N;

P10Q+K32E+A34L+I205T;

P10Q+K32E+A34L+V253N;

P10Q+K32E+A34L+T106N+I205T;

P10Q+K32E+A34L+T106N+V253N;

P10Q+K32E+A34L+I205T+V253N;

P10Q+K32E+A34L+T106N+I205T+V253N;

P10Q+K32E+A34L+R36E+T106N;

P10Q+K32E+A34L+R36E I205T;

P10Q+K32E+A34L+R36E+V253N;

P10Q+K32E+A34L+R36E+T106N+I205T;

P10Q+K32E+A34L+R36E+T106N+V253N;

P10Q+K32E+A34L+R36E+I205T+V253N; and

P10Q+K32E+A34L+R36E+T106N+I205T+V253N.

Preferred combined variants having multiple substitutions in the Gla domain and at least one introduced N-glycosylation site include:

P10Q+K32E+T106N;

P10Q+K32E+I205T;

P10Q+K32E+V253N;

P10Q+K32E+T106N+I205T;

P10Q+K32E+T106N+V253N;

P10Q+K32E+I205T+V253N;

P10Q+K32E+A34E+R36E+T106N;

P10Q+K32E+A34E+R36E+I205T;

P10Q+K32E+A34E+R36E+V253N;

P10Q+K32E+A34E+R36E+T106N+I205T;

P10Q+K32E+A34E+R36E+T106N+V253N; and

P10Q+K32E+A34E+R36E+I205T+V253N.

In addition, any of the combined variants listed above may, if desired, include the insertion A3AY.

Variants with Modifications in the Tissue Factor Binding Site

In another embodiment, the methods of the invention may be performed with a FVII or FVIIa variant comprising a substitution in at least one position selected from the group consisting of L39, I42, S43, K62, L65, F71, E82 and F275. These amino acid substitutions in the tissue factor (TF) binding site of the FVII molecule result in an improved clotting activity.

Preferred substitutions in these positions in the TF binding site include the following:
L39E, L39Q or L39H
I42R
S43Q
K62E or K62R
L65Q or L65S
F71D, F71E, F71N, F71Q or F71Y
E82Q or E82N
F275H

The variant of this embodiment may comprise one of the above-mentioned substitutions in the TF binding site, or it may comprise more than one such substitution, e.g. two or three of the above-listed substitutions. In one preferred embodiment, the FVII or FVIIa variant comprises a substitution selected from the group consisting of S43Q, K62E, L65Q and F71Y, in particular selected from the group consisting of S43Q, K62E and L65Q.

Preferably, variants according to the present embodiment with one or more modifications in the TF binding site as defined above are not selected from among the following:

[K32E+D33N+A34T+K38T+L39E]hFVII;

[A1Y+K32E+D33N+A34T+K38T+L39E]hFVII;

[A1Y+A3S+F4GK+K32E+D33N+A34T+K38T+L39E]hFVII;

[A1Y+L8F+R9V+P10Q+K32E+D33N+A34T+K38T+L39E]hFVII;

[A1Y+A3S+F4GK+L8F+R9V+P10Q+K32E+D33N+A34T+K38T+L39E]hFVII;

[A3S+F4GK+K32E+D33N+A34T+K38T+L39E]hFVII;

[A3S+F4GK+L8F+R9V+P10Q+K32E+D33N+A34T+K38T+L39E]hFVII;

[L8F+R9V+P10Q+K32E+D33N+A34T+K38T+L39E]hFVII;

[I42N]hFVII/hFVIIa; [I42S]hFVII/hFVIIa; [I42A]hFVII/hVIIa; [I42Q]hFVII/hVIIa.

Further information about variants of this type having modifications in the TF binding site may be found in WO 2004/029091.

It will be understood that these substitutions in the TF binding site may if desired be combined with one or more of the other types of modifications described elsewhere herein, e.g. the modifications in the Gla domain as described above, introduction of at least one in vivo N-glycosylation site, and/or conjugation with a PEG polymer as described below.

Variants having an Introduced PEGylation Site

In a further embodiment, the methods of the invention may be performed with a FVII or FVIIa variant having at least one polymer molecule, in particular a polyethylene glycol (PEG) or other polyalkylene oxide, conjugated to an attachment group selected from the group consisting of a lysine residue, a cysteine residue, an aspartic acid residue, a glutamic acid residue, a histidine residue, and a tyrosine residue, preferably a cysteine or a lysine residue.

Methods for conjugating various polypeptides with a polyethylene glycol moiety (“PEGylation”) are known in the art. For example, WO 01/58935 describes methods by which PEG moieties may be attached to a FVII or FVIIa variant which has been modified relative to hFVII so as to have at least one introduced and/or removed attachment site for PEGylation, for example one or more introduced lysine residues, optionally in combination with removal of one or more lysine residues in positions where PEGylation is not desired, or one or more introduced cysteine residues, in this case optionally in combination with removal of one or more cysteine residues. As described in WO 01/58935, introduction of amino acid residues for PEG conjugation, e.g. a lysine or cysteine residue, preferably takes place in positions where the amino acid residue in the wild-type HFVII sequence has at least 25% of its side chain exposed to the surface, preferably at least 50%.

WO 02/02764 discloses vitamin K-dependent polypeptides such as FVIIa linked to a PEG polymer, for example wild-type human FVIIa and a variant of FVIIa having the substitutions P10Q and K32E.

Various PEGylation technologies are known in the art, and depending on the nature and the degree of PEGylation desired, persons skilled in the art will be able to select a suitable PEGylation technology to attach PEG polymers on one or more desired amino acid residue. For example, amine-specific activated PEG derivatives preferentially attach to the N-terminal amino group or the •-amino groups of lysine residues via an amide bond. Examples of amine-specific activated PEG derivatives include mPEG-succinimidyl propionate (mPEG-SPA), mPEG-succinimidyl butanoate (mPEG-SBA) and mPEG-succinimidyl •-methylbutanoate (mPEG-SMB) (available from Nektar Therapeutics; see the Nektar Advanced PEGylation Catalog 2004, “Polyethylene Glycol and Derivatives for Advanced PEGylation”); and PEG-SS (Succinimidyl Succinate), PEG-SG (Succinimidyl Glutarate), PEG-NPC (p-nitrophenyl carbonate), and PEG-isocyanate, available from SunBio. Activated PEG derivatives are also available e.g. from NOF Corporation, Japan. If desired, conditions can be adapted using certain amine-specific activated PEG derivatives to obtain N-terminal specific PEGylation. For example, WO 96/11953 describes methods for preparing N-terminally PEGylated proteins.

Similarly, activated PEG derivatives are available for sulfhydryl-selective attachment to a cysteine residue. Examples of such sulfhydryl-selective activated PEG derivatives are mPEG-Maleimide (mPEG-MAL), PEG2-Maleimide (mPEG2-MAL), and mPEG-Vinyl Sulfone (mPEG-VS), available from Nektar Therapeutics.

The PEGylation will be designed in each case so as to produce the optimal molecule with respect to the number of PEG molecules attached, the size and form of such molecules (e.g. whether they are linear or branched), and the attachment site(s) in the polypeptide. The molecular weight of the polymer to be used may e.g. be chosen on the basis of the desired effect to be achieved. For instance, if the primary purpose of the conjugation is to achieve a conjugate having a high molecular weight (e.g. to reduce renal clearance and thus improve circulation half-life) it may be desirable to conjugate one or a few relatively high molecular weight polymer molecules as possible to obtain the desired molecular weight. When a high degree of shielding is desirable this may be obtained by use of a sufficiently high number of low molecular weight polymer molecules (e.g. with a molecular weight of from about 300 Da to about 5 kDa) to effectively shield all or most protease cleavage sites or other vulnerable sites of the polypeptide. For instance, 2-8, such as 3-6 such polymers may be used.

In connection with conjugation to only a single attachment group on the protein (e.g. the N-terminal amino group), it may be advantageous that the polymer molecule, which may be linear or branched, has a high molecular weight, preferably about 10-25 kDa, such as about 15-25 kDa, e.g. about 20 kDa. For example, N-terminal PEGylation using e.g. mPEG-SPA with a molecular weight of 20,000 can be performed substantially as described in WO 02/02764. Where it is desired to attach two or more PEG polymers to each polypeptide, e.g. up to 3, 4, 5, 6, 7 or 8 PEG polymers, such as 2-6 or 3-5 PEG polymers, the PEG will typically have a somewhat lower molecular weight, e.g. about 2-15 kDa, such as about 4-12 kDa, e.g. about 5 kDa or 10 kDa.

It will further be understood that the FVII or FVIIa variants for use in the methods of the invention may, in addition to the attachment of one or more PEG polymers, also include one or more of the amino acid modifications otherwise described herein to provide e.g. an increased phospholipid membrane binding affinity and/or an increased tissue factor independent activity, and/or to provide one or more introduced in vivo N-glycosylation sites.

### Other Modifications

In a further embodiment, variants for use in the methods of the invention may comprise, in addition to one or more of the modifications described above, at least one further amino acid substitution in a position selected from the group consisting of position 74, 77 and 116, in particular P74S, E77A and/or E116D.

In a still further embodiment, the FVII or FVIIa variant may contain mutations known to increase the intrinsic activity of the polypeptide, for example those described in WO 02/22776. For example, the variant may comprise at least one modification in a position selected from the group consisting of 157, 158, 296, 298, 305, 334, 336, 337 and 374. Examples of such substitutions include one or more of V158D, E296D, M298Q, L305V and K337A.

In another embodiment, the FVII or FVIIa variant may also contain other mutations such as the substitution K341Q disclosed by Neuenschwander et al, Biochemistry, 1995; 34:8701-8707. Other possible additional substitutions include D196K, D196N, G237L, G237GAA and combinations thereof.

### Preferred Activity

In one preferred embodiment of the invention, the polypeptide variant, in its activated form and when compared to a reference molecule such as rhFVIIa, has an increased FX activation activity, in particular when assayed in a tissue factor-independent assay, such as the “TF-independent Factor X Activation Assay” disclosed herein. More particularly, it is preferred that the ratio between the FX activation activity of the polypeptide variant, in its activated form, and the FX activation activity of a reference molecule is at least about 2, such as at least about 3, 4 or 5, e.g. at least about 10.

In another preferred embodiment, the variants possess an increased clotting activity (i.e. a reduced clotting time) as compared to rhFVIIa, in particular a ratio between the time to reach clot formation for the variant (tᵥₐᵣᵢₐₙₜ) and the time to reach clot formation for rhFVIIa (t_{wt}) of at the most 0.9 when assayed in the “Whole Blood Assay” described herein. Preferably, this ratio is at the most 0.8, such as at the most 0.7, more preferably at the most 0.6, still more preferably at the most 0.5, such as at the most 0.4.

Further information regarding variants having an increased FX activation activity and an increased clotting activity may be found in WO 03/093465 and WO 2004/111242.

### Pharmaceutical Composition of the Invention and its Use

As indicated above, a further aspect the invention relates to use of the polypeptide variants described herein for the manufacture of a medicament for the treatment of the conditions described above wherein clot formation is desirable.

The polypeptide variants are administered to patients in an “effective amount” or “therapeutically effective dose”, which may in some cases approximately parallel that employed in therapy with rFVIIa such as NovoSeven®, but will often be a somewhat lower dosage in view of the generally increased efficacy of the variants described herein compared to rFVIIa. By “effective amount” or “therapeutically effective dose” herein is meant a dose that is sufficient to produce the desired effects in relation to the condition for which it is administered, in particular an amount of a FVII or FVIIa variant that is effective to stop or prevent the unwanted bleeding or to reduce the bleeding to an acceptable level. The exact dose will depend on the circumstances, e.g. the condition being treated, the administration schedule, whether the polypeptide variant is administered alone or in conjunction with other therapeutic agents, the plasma half-life of the variant, and the general health of the patient.

For purposes of the present invention, it is contemplated that the FVII or FVIIa variant will be administered as single or multiple injections (bolus or transfusion), in doses ranging from about 10 to about 600 μg/kg body weight, typically from about 20 to about 300 μg/kg, preferably from about 25 to about 150 μg/kg, for example from about 40 to about 120 μg/kg.

The polypeptide variant is preferably administered in a composition including one or more pharmaceutically acceptable carriers or excipients. “Pharmaceutically acceptable” means a carrier or excipient that does not cause any untoward effects in patients to whom it is administered. Such pharmaceutically acceptable carriers and excipients as well as suitable pharmaceutical formulation methods are well known in the art (see, for example, Remington's Pharmaceutical Sciences, 19th edition, A. R. Gennaro, Ed., Mack Publishing Company [1995]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]).

The polypeptide variant can be used “as is” and/or in a salt form thereof. Suitable salts include, but are not limited to, salts with alkali metals or alkaline earth metals, such as sodium, potassium, calcium and magnesium, as well as e.g. zinc salts. These salts or complexes may by present as a crystalline and/or amorphous structure.

The pharmaceutical composition may be administered alone or in conjunction with other therapeutic agents. These agents may be incorporated as part of the same pharmaceutical composition or may be administered separately from the polypeptide variant, either concurrently or in accordance with another treatment schedule.

A “patient” for the purposes of the present invention includes both humans and other mammals. Thus, the methods are applicable to both human therapy and veterinary applications, in particular to human therapy.

The pharmaceutical composition comprising the polypeptide variant may be formulated in a variety of forms, e.g. as a liquid, gel, lyophilized, or as a compressed solid. The preferred form will depend upon the particular indication being treated and will be apparent to one skilled in the art.

In particular, the pharmaceutical composition comprising the polypeptide variant may be formulated in lyophilised or stable soluble form, or in a stable liquid formulation, typically an aqueous formulation. The polypeptide variant may be lyophilised by a variety of procedures known in the art. The polypeptide variant may be in a stable soluble form by the removal or shielding of proteolytic degradation sites as described herein. The advantage of obtaining a stable soluble preparation lies in easier handling for the patient and, in the case of emergencies, quicker action, which potentially can become life saving. The preferred form will depend upon the particular indication being treated and will be apparent to one of skill in the art.

The administration of the formulations of the present invention can be performed in a variety of ways, including, but not limited to, orally, subcutaneously, intravenously, intracerebrally, intranasally, transdermally, intraperitoneally, intramuscularly, intrapulmonary, vaginally, rectally, intraocularly, or in any other acceptable manner. The formulations can be administered continuously by infusion, although bolus injection is acceptable, using techniques well known in the art, such as pumps or implantation. In some instances the formulations may be directly applied as a solution or spray.

### Parentals

A preferred example of a pharmaceutical composition is a solution, in particular an aqueous solution, designed for parenteral administration. Although in many cases pharmaceutical solution formulations are provided in liquid form, appropriate for immediate use, such parenteral formulations may also be provided in frozen or in lyophilized form. In the former case, the composition must be thawed prior to use. The latter form is often used to enhance the stability of the active compound contained in the composition under a wider variety of storage conditions, as it is recognized by those skilled in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

In case of parenterals, they are prepared for storage as lyophilized formulations or aqueous solutions by mixing, as appropriate, the polypeptide variant having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic surfactants or detergents, antioxidants, and/or other miscellaneous additives such as bulking agents or fillers, chelating agents, antioxidants and cosolvents.

Detailed information on parental formulations suitable for administration of FVII variants, as well as sustained release preparations, is found in WO 01/58935 and WO 03/093465, incorporated herein by reference.

### Assay Methods

The following in vitro assays are suitable for determining the clotting activity and other properties of hFVIIa and variants thereof.

### Measurement of Reduced Sensitivity to Proteolytic Degradation

Proteolytic degradation can be measured using the assay described in U.S. Pat. No. 5,580,560, Example 5, where proteolysis is autoproteolysis. Furthermore, reduced proteolysis can be tested in an in vivo model using radiolabelled samples and comparing proteolysis of rhFVIIa and the polypeptide variant of the invention by withdrawing blood samples and subjecting these to SDS-PAGE and autoradiography.

Irrespective of the assay used for determining proteolytic degradation, “reduced proteolytic degradation” is intended to mean a measurable reduction in cleavage compared to that obtained by rhFVIIa as measured by gel scanning of Coomassie stained SDS-PAGE gels, HPLC or as measured by conserved catalytic activity in comparison to wild type using the tissue factor independent activity assay described below.

### Determination of the Molecular Weight of Polypeptide Variants

The molecular weight of polypeptide variants is determined by either SDS-PAGE, gel filtration, Western Blots, matrix assisted laser desorption mass spectrometry or equilibrium centrifugation, e.g. SDS-PAGE according to Laemmli, U. K., Nature Vol 227 (1970), pp. 680-85.

### Determination of Phospholipid Membrane Binding Affinity

Phospholipid membrane binding affinity may be determined as described in Nelsestuen et al., Biochemistry, 1977; 30;10819-10824, or as described in Example 1 in U.S. Pat. No. 6,017,882.

### TF-Independent Factor X Activation Assay

This assay has been described in detail on page 39826 in Nelsestuen et al., J Biol Chem, 2001; 276:39825-39831.

Briefly, the molecule to be assayed (either hFVIIa, rhFVIIa or a polypeptide variant in its activated form) is mixed with a source of phospholipid (preferably phosphatidylcholine and phosphatidylserine in a ratio of 8:2) and relipidated Factor X in Tris buffer containing BSA. After a specified incubation time the reaction is stopped by addition of excess EDTA. The concentration of factor Xa is then measured from absorbance change at 405 nm after addition of a chromogenic substrate (S-2222, Chromogenix). After correction for background the tissue factor independent activity of rhFVIIa (a_{wt}) is determined as the absorbance change after 10 minutes and the tissue factor independent activity of the polypeptide variant of the invention (aᵥₐᵣᵢₐₙₜ) is also determined as the absorbance change after 10 minutes. The ratio between the activity of the polypeptide variant, in its activated form, and the activity of rhFVIIa is defined as aᵥₐᵣᵢₐₙₜ/a_{wt}.

### Clotting Assay

The clotting activity of the FVIIa and variants thereof are measured in one-stage assays and the clotting times are recorded on a Thrombotrack IV coagulometer (Medinor). Factor VII-depleted human plasma (American Diagnostica) is reconstituted and equilibrated at room temperature for 15-20 minutes. 50 μl of plasma are then transferred to the coagulometer cups. FVIIa and variants thereof are diluted in Glyoxaline Buffer (5.7 mM barbiturate, 4.3 mM sodium citrate, 117 mM NaCl, 1 mg/ml BSA, pH 7.35). The samples are added to the cup in 50 μl and incubated at 37° C. for 2 minutes. Thromboplastin (Medinor) is reconstituted with water and CaCl₂ is added to a final concentration of 4.5 mM. The reaction is initiated by adding 100 •1 thromboplastin. To measure the clotting activity in the absence of TF the same assay may be used without addition of thromboplastin. Data are analysed using PRISM software.

### Whole Blood Assay

The clotting activity of FVIIa and variants thereof are measured in one-stage assays and the clotting times are recorded on a Thrombotrack IV coagulometer (Medinor). 100 μl of FVIIa or variants thereof are diluted in a buffer containing 10 mM glycylglycine, 50 mM NaCl, 37.5 mM CaCl₂, pH 7.35 and transferred to the reaction cup. The clotting reaction is initiated by addition of 50 μl blood containing 10% 0.13 M tri-sodium citrate as anticoagulant. Data are analysed using Excel or PRISM software.

### Amidolytic Assay

The ability of the variants to cleave small peptide substrates can be measured using the chromogenic substrate S-2288 (D-Ile-Pro-Arg-p-nitroanilide). FVIIa is diluted to about 10-90 nM in assay buffer (50 mM Na-Hepes pH 7.5, 150 mM NaCl, 5 mM CaCl₂, 0.1% BSA, 1U/ml Heparin). Furthermore, soluble TF (sTF) is diluted to 50-450 nM in assay buffer. 120 μl of assay buffer is mixed with 20 μl of the FVIIa sample and 20 μl sTF. After 5 min incubation at room temperature with gentle shaking, followed by 10 min incubation at 37° C., the reaction is started by addition of the S-2288 substrate to 1 mM and the absorption at 405 nm is determined at several time points.

### ELISA Assay

FVII/FVIIa (or variant) concentrations are determined by ELISA. Wells of a microtiter plate are coated with an antibody directed against the protease domain using a solution of 2 μg/ml in PBS (100 μl per well). After overnight coating at R.T. (room temperature), the wells are washed 4 times with THT buffer (100 mM NaCl, 50 mM Tris-HCl pH 7.2 0.05% Tween-20). Subsequently, 200 μl of 1% Casein (diluted from 2.5% stock using 100 mM NaCl, 50 mM Tris-HCl pH 7.2) is added per well for blocking. After 1 hr incubation at R.T., the wells are emptied, and 100 μl of sample (optionally diluted in dilution buffer (THT+0.1% Casein)) is added. After another incubation of 1 hr at room temperature, the wells are washed 4 times with THT buffer, and 100 μl of a biotin-labelled antibody directed against the EGF-like domain (1 μg/ml) is added. After another 1 hr incubation at R.T., followed by 4 more washes with THT buffer, 100 μl of streptavidin-horse radish peroxidase (DAKO A/S, Glostrup, Denmark, 1/10000 diluted) is added. After another 1 hr incubation at R.T., followed by 4 more washes with THT buffer, 100 μl of TMB (3,3′,5,5′-tetramethylbenzidine, Kem-en-Tech A/S, Denmark) is added. After 30 min incubation at R.T. in the dark, 100 μl of 1 M H₂SO₄ is added and OD_{450 nm} is determined. A standard curve is prepared using rhFVIIa (NovoSeven®).

Alternatively, FVII/FVIIa or variants may be quantified through the Gla domain rather than through the protease domain. In this ELISA set-up, wells are coated overnight with an antibody directed against the EGF-like domain and for detection, a calcium-dependent biotin-labelled monoclonal anti-Gla domain antibody is used (2 μg/ml, 100 μl per well). In this set-up, 5 mM CaCl₂ is added to the THT and dilution buffers.

### Thrombogram Assay

The effect of hFVIIa, rhFVIIa or FVIIa variants on thrombin generation in human plasma is tested in a modified version of the assay described on page 589 in Hemker et al. (2000) Thromb Haemost 83:589-91. Briefly, the molecule to be assayed (either hFVIIa, rhFVIIa or a variant) is mixed with FVII-depleted platelet poor plasma (PPP) containing either relipidated recombinant tissue factor (such as Innovin from Dade Behring) or phospholipid (phosphatidylcholine and phosphatidylserine in a ratio of 8:2, or phosphatidylcholine, phosphatidylserine and phosphatidylethanol in a ratio of 4:2:4).

The reaction is started by addition of a fluoregenic thrombin substrate and calcium chloride. The fluorescence is measured continuously and the thrombin amidolytic activity is determined by calculating the slope of the fluorescence curve (the increase in fluorescence over time). In this way the time until maximum thrombin amidolytic activity is obtained (Tₘₐₓ), and the thrombin generation rate (maximal increase in thrombin activity) and the total thrombin work (area under the curve (AUC)) can be calculated.

Frozen citrated FVII-depeleted plasma is thawed in the presence of corn trypsin inhibitor (100 μg/ml serum) to inhibit the contact pathway of coagulation. To each well of a 96-well microtiter plate is added 80 μl plasma and 20 μl buffer containing rhFVII or variant to be tested in final concentrations of between 0.1 and 100 nM. Recombinant human tissue factor (rTF) is added in 5 μl assay buffer to a final concentration of 1 pM. The assay buffer consists of 20 mM Hepes, 150 mM NaCl and 60 mg/ml BSA in distilled water. The reaction is started by adding 20 μl of the substrate solution containing 0.1 M calcium chloride. The assay plate and reagents are pre-warmed to 37° C. and the reaction takes place at this temperature. The fluorimeter is e.g. a BMG Fluorimeter with an excitation filter at 390 nm and an emission filter at 460 nm. The fluorescence is measured in each well of 96-well clear bottom plates at 20-40 second intervals over 30-180 minutes. Data are analyzed using PRISM Software.

### Tissue Factor Binding Surface Plasmon Resonance Assay (Biacore Assay)

Surface plasmon resonance analysis is used to determine the relative binding of wild-type Factor VIIa and variants thereof to soluble tissue factor. Recombinant soluble tissue factor that contains the extracellular domain iss coupled to 270 response units on a Biacore CM5 chip using NHS/EDC coupling. Soluble tissue factor is coupled at pH 4.5 to enable interaction with the chip surface.

In this assay, tissue factor binding of factor VII protein is compared at a single concentration of FVIIa or variant to allow a relative comparison of the variants to wild-type. This concentration is determined by means of a standard curve of wild type FVIIa that is flowed over the chip in concentrations between 75 and 0 μg/ml. FVIIa is removed by addition of 10 mM EDTA. A concentration of 15 μg/ml is suitable to give binding in the linear range. Variants of FVIIa are flowed over the chip at 15 μg/ml to determine the relative binding strength of FVIIa or variants to tissue factor.

1. A method for treatment of bleeding in a patient, wherein the bleeding is associated with trauma, intracerebral hemorrhage (ICH), traumatic brain injury (TBI), burns, variceal bleeds, gastrointestinal bleeding, surgical bleeds, transplantation, fibrinolytic treatment, anticoagulant treatment, postpartum hemorrhage, viral-induced hemorrhage, thrombocytopenia, or factor deficiency, comprising administering to said patient a variant of a human FVII or FVIIa polypeptide that differs from SEQ ID NO:1 in 1-15 amino acid residues and has an increased phospholipid membrane binding affinity and/or an increased tissue factor independent activity relative to hFVIIa.

2. The method of claim 1, wherein the polypeptide variant comprises at least one amino acid modification in the Gla domain.

3. The method of claim 2, wherein the polypeptide variant comprises at least one amino acid substitution in a position selected from the group consisting of residues 10, 11, 28, 32, 33, 34, 36 and 38.

4. The method of claim 3, wherein the polypeptide variant comprises a substitution of a glutamine, a glutamic acid, an aspartic acid or an asparagine residue in position 10.

5. The method of claim 3, wherein the polypeptide variant comprises a substitution of a glutamic acid or an aspartic acid residue in position 32.

6. The method of claim 3, wherein the polypeptide variant comprises the substitutions P10Q+K32E.

7. The method of claim 1, wherein the polypeptide variant comprises at least one substitution selected from the group consisting of R28E/F, D33I3/LM/V/F/Y/W, A34E/D/I/L/MIVN/FY/W, R36E/D, and K38E/D.

8. The method of claim 7, wherein the polypeptide variant comprises the substitution A34E/L and/or R36E.

9. The method of claim 1, wherein the polypeptide variant comprises an insertion of at least one amino acid residue between position 3 and 4.

10. The method of claim 9, wherein the polypeptide variant comprises the insertion A3AY.

11. The method of claim 1, wherein the polypeptide variant comprises at least one introduced in vivo N-glycosylation site relative to SEQ ID NO:1.

12. The method of claim 11, wherein the polypeptide variant comprises up to five introduced in vivo N-glycosylation sites relative to SEQ ID NO:1.

13. The method of claim 11, wherein the polypeptide variant comprises at least one introduced in vivo N-glycosylation site created by a substitution selected from the group consisting of A51N, G58N, T106N, K109N, G124N, K143N+N145T, A175T, I205S, I205T, V253N, T267N, T267N+S269T, S314N+K316S, S314N+K316T, R315N+V317S, R315N+V317T, K316N+G318S, K316N+G318T, G318N and D334N.

14. The method of claim 13, wherein the polypeptide variant comprises at least one introduced in vivo N-glycosylation site created by a substitution selected from the group consisting of T106N, I205T and V253N.

15. The method of claim 14, wherein the polypeptide variant comprises two introduced in vivo N-glycosylation sites created by a substitution selected from the group consisting of T106N, I205T and V253N.

16. The method claim 1, wherein the polypeptide variant comprises a substitution in at least one position selected from the group consisting of L39, I42, S43, K62, L65, F71, E82 and F275.

17. The method claim 1, wherein the polypeptide variant is conjugated to at least one polyethylene glycol (PEG) polymer.

18. The method of claim 17, wherein the polypeptide variant is conjugated to up to eight PEG polymers.

19. The method of claim 17, wherein the polypeptide variant comprises at least one PEG polymer conjugated to a lysine residue or the N-terminal.

20. The method of claim 17, wherein the polypeptide variant comprises at least one PEG polymer conjugated to a cysteine residue.

21. The method of claim 1, wherein the polypeptide variant is in its activated form.

22. The method of claim 1, wherein the ratio between the activity of the polypeptide variant, in its activated form, and the activity of rhFVIIa is at least about 2 when assayed in the “TF-independent Factor X Activation Assay”.

23. The method of claim 1, wherein the ratio between the activity of the polypeptide variant, in its activated form, and the activity of rhFVIIa is at the most 0.9 when assayed in the “Whole Blood Assay”.

24. The method of claim 1, wherein the polypeptide is administered in the activated form.

25. The method of claim 1, wherein the polypeptide is administered in the non-activated form.

26. (canceled)
